(19) United States
(12) Patent Application Publication
(10) Pub. No.: US 2026/0108348 A1
(43) Pub. Date: Apr. 23, 2026

(54) DEVICES AND METHODS FOR LENS DELIVERY

(71) Applicant: Johnson & Johnson Surgical Vision, Inc., Irvine, CA (US)

(72) Inventors: David RUDDOCKS, Celina, TX (US); Jason SAN JOSE, Foothill Ranch, CA (US); Vanessa CORRALES-YANG, Mission Viejo, CA (US); Jeremy HEMINGWAY, Irvine, CA (US); Hessein ALI, Irvine, CA (US); Ehsan RAJABI, Irvine, CA (US)

(21) Appl. No.: 19/352,852

(22) Filed: Oct. 8, 2025

Related U.S. Application Data
(60) Provisional application No. 63/708,526, filed on Oct. 17, 2024.

(51) Int. Cl. A61F 2/16

(52) U.S. Cl. CPC A61F 2/167

(57) ABSTRACT

An example lens delivery device is provided. The lens delivery device can include a body comprising a channel therethrough. A first interference portion can be located within the channel. A plunger can be configured to receive a user-supplied force to move the plunger axially through the channel of the body. The plunger can include a second interference portion, located along a length of the plunger, configured to interact with the first interference portion during the axial movement of the plunger to form a damping mechanism to maintain a specified resistance to the user-supplied force.

### CROSS-REFERENCE TO RELATED APPLICATIONS

This application claims the benefit under 35 U.S.C. § 119(e) of U.S. Provisional Patent Application No. 63/708,526, filed Oct. 17, 2024, which is incorporated herein by reference in its entirety.

### FIELD

The present invention generally relates to devices and methods for lens delivery. More specifically, certain embodiments relate to devices and methods for delivering and inserting an intraocular lens into a patient's eye.

### BACKGROUND

Devices and methods for delivering an implantable lens, such as an intraocular lens (IOL), are typically configured to apply the force required to advance the lens through a delivery device and into an eye of a patient. Certain devices and methods utilize a push system in which the lens is advanced through cartridge by a pushing force applied by the surgeon to a plunger system. As the lens is advanced into the cartridge, the lens is forced to compress through a relatively narrow tip, which provides a force against the plunger. However, once the largest cross-section of the lens passes through the cartridge, the lens no longer provides a constraint against the plunger system. The abrupt removal of the counter force can lead to unwanted force from the plunger system on the lens. In particular, there may be insufficient time between the removal of the force and the surgeon's reaction to remove the force applied to the plunger system. The surgeon's continued application of force, in the absence of the counter force from the lens, can lead to reduced control for the delivery of the lens into the patient's eye.

Accordingly, alternative devices and methods for lens delivery would be useful.

### SUMMARY

The present invention is directed to devices and methods for lens delivery.

An example lens delivery device is provided. The lens delivery device can include a body comprising a channel therethrough. A first interference portion can be located within the channel. A plunger can be configured to receive a user-supplied force to move the plunger axially through the channel of the body. The plunger can include a second interference portion, located along a length of the plunger, configured to interact with the first interference portion during the axial movement of the plunger to form a damping mechanism to maintain a specified resistance to the user-supplied force.

A system for delivering an intraocular lens (IOL) to a patient's eye is provided. The system can include a modular cartridge containing the IOL. The system can further include a lens delivery device that can be configured to receive the modular cartridge. The lens delivery device can include a body comprising a channel therethrough. A first interference portion can be located within the channel. A plunger can be configured to receive a user-supplied force to move the plunger axially through the channel of the body. The plunger can include a second interference portion, located along a first length of the plunger, configured to interact with the first interference portion during the axial movement of the plunger to form a damping mechanism to maintain a specified resistance to the user-supplied force. The axial movement of the plunger can be configured to drive the IOL through the modular cartridge coupled to the body, delivering the lens from the lens delivery device.

A method for delivering a lens is provided. The method can include providing a lens delivery device. The lens delivery device can include a body comprising a channel therethrough. A first interference portion can be located within the channel. A plunger can be configured to receive a user-supplied force to move the plunger axially through the channel of the body. The plunger can include a second interference portion, located along a length of the plunger, configured to interact with the first interference portion during the axial movement of the plunger to form a damping mechanism to maintain a specified resistance to the user-supplied force. The method can further include coupling a modular cartridge containing a lens to the body. The axial movement of the plunger can be configured to drive the lens through modular cartridge, delivering the lens from the lens delivery device. The method can further include providing the user-supplied force to move the plunger axially through the channel of the body to eject the lens from the modular cartridge.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is a side cross-sectional view of an example system for delivering an intraocular lens (IOL) to a patient's eye including an example lens delivery device according to aspects of the present invention.

FIG. 2A is a perspective view an example nut used in a lens delivery device according to aspects of the present invention.

FIG. 2B is a perspective view an example nut used in a lens delivery device according to aspects of the present invention.

FIG. 2C is a cross-sectional perspective view of an example nut used in a lens delivery device including an example interference portion therein according to aspects of the present invention.

FIG. 3A is a perspective view of an example plunger used in a lens delivery device according to aspects of the present invention.

FIG. 3B is a side view an example plunger used in a lens delivery device according to aspects of the present invention.

FIG. 4A is a side cross-sectional view of another example system for delivering an IOL to a patient's eye including another example lens delivery device according to aspects of the present invention.

FIG. 4B is a perspective view of an example nut used in a lens delivery device including an example interference portion therein according to aspects of the present invention.

FIG. 4C is a cross-sectional perspective view of an example nut used in a lens delivery device including an example interference portion therein according to aspects of the present invention.

FIG. 5A perspective view of an example plunger used in a lens delivery device according to aspects of the present invention.

FIG. 5B is a side view an example plunger used in a lens delivery device according to aspects of the present invention.

FIG. 6A is a side cross-sectional view of another example system for delivering an IOL to a patient's eye including another example lens delivery device according to aspects of the present invention.

FIG. 6B is a perspective view of an example nut used in a lens delivery device including an example interference portion therein according to aspects of the present invention.

FIG. 6C is a cross-sectional perspective view example nut used in a lens delivery device including an example interference portion therein according to aspects of the present invention.

FIG. 7 is a flowchart of an exemplary method for lens delivery according to aspects of the present invention.

### DETAILED DESCRIPTION

The example devices and methods described herein generally involve providing delivery of an implantable lens, such as an intraocular lens (IOL), using a lens delivery device that allows a surgeon to deliver the lens through a modular cartridge using a pushing force. The application of the pushing force causes the lens to be constrained in the insertion tip of the modular cartridge creating a resistance against the applied pushing force. The disclosed example devices and methods provide improvements in lens delivery by providing a damping mechanism that counterbalances the removal of the resistance force provided by the constrained lens when the largest portion of the lens passes through the insertion tip.

The example devices and methods disclosed herein provide for improvement in the delivery of the lens by reducing the abrupt change in force to allow for a more controlled delivery of the lens. The disclosed devices and methods therefore reduce the risk of damage to the lens or the associated haptics during lens delivery.

Various example devices and methods are presented herein. Features from each example are combinable with other examples as understood by persons skilled in the pertinent art.

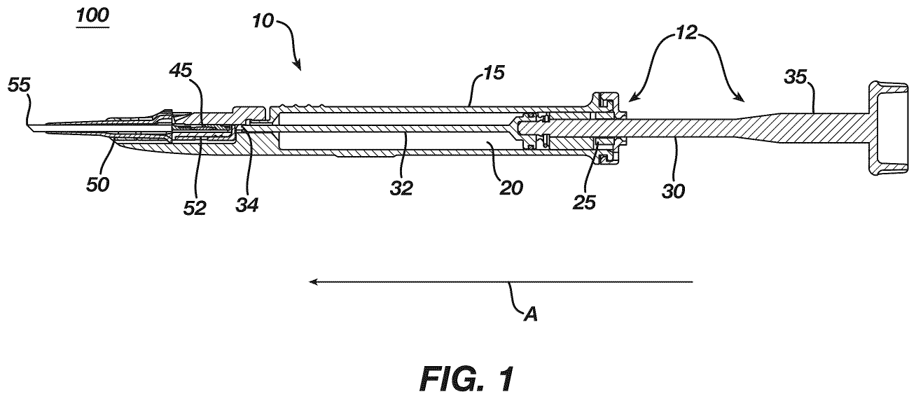

FIG. 1 is an illustration of an example system 100 for delivering an intraocular lens (IOL), such as lens 45, to a patient's eye including an example lens delivery device 10 and a modular cartridge 50. In this embodiment, the lens delivery device 10 includes a dampening mechanism 12 that is configured to maintain a specified resistance to the user-supplied force required to provide delivery of the lens 45, as described in further detail below. The system 100 further includes the modular cartridge 50 configured to hold the lens 45, such as an IOL, in different positions throughout the delivery process, as further discussed below. The lens delivery device 10 is configured to receive the modular cartridge 50 thereon, as shown in FIG. 1. The modular cartridge 50 can include a lens storage portion 52 that holds lens 45 and an insertion tip 55. The insertion tip 55 can include a tapered portion that causes lens 45 to be constrained therein creating a force during delivery of the lens 45. As described in further detail below, the dampening mechanism 12 is configured to prevent uncontrolled delivery of the lens 45 after the largest cross-section of the lens 45 passes through the insertion tip 55 of the modular cartridge 50. The dampening mechanism 12, as described in further detail below, can be designed to modulate the lens delivery based on the size of the lens 45 to be employed in the lens delivery device 10.

The lens delivery device 10 includes a body 15 comprising a channel 20 therethrough. The body 15 provides an external shell of lens delivery device 10 and can in some examples be formed of a disposable material, such as a plastic. Alternatively, the body 15 can be made of a material that can be sterilized and reused. The channel 20 extends through the body and is configured to house various elements for the delivery of the lens 45. The body 15 is further configured to receive the modular cartridge 50 thereon, as shown for example in FIG. 1.

In this embodiment, the channel 20 of the body 15 is configured to receive a plunger 30 therein. The plunger 30 is configured to receive a user-supplied force to move the plunger 30 axially, along the axis A shown in FIG. 1, through the channel 20 of the body 15. A user, e.g., a surgeon or physician, can push against the plunger 30 to apply the required force to advance the implantable lens 45, e.g., an IOL, to be ejected from the system 100, as described in further detail below. In this example, the plunger 30, when moved axially in the channel 20, applies a force to a pushrod 32, which includes a pushrod tip 34 that is configured to apply the required force to the lens 45 for lens delivery. The axial movement of the plunger 30 along axis A is configured to drive the lens 45 through the modular cartridge 50, including insertion tip 55, delivering the lens 45 from the lens delivery device 10. The dampening mechanism 12 is configured to provide a force counter to the user-supplied force to slow the axial movement of the plunger 30 based, at least, on the user-supplied force.

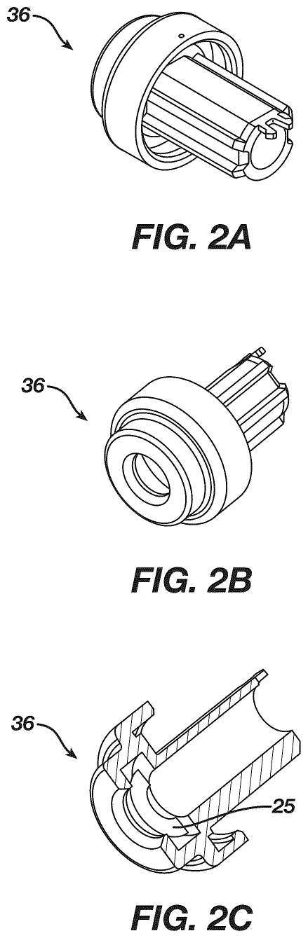

In this example, the dampening mechanism 12 includes a first interference portion 25 located within the channel 20 and a second interference portion 35 that is part of the plunger 30, as described in further detail below. Alternatively, the second interference portion 35 could be provided by a sleeve located around the plunger 30 that provides an increased diameter. In one example, the first interference portion 25 is located in a nut 36, as shown for example in FIGS. 2A-2C, 4A-4C, and 6A-6C, that may be positioned within the channel 20 of the body 15 of the lens delivery device 10, although the first interference portion 25 can be located directly in the channel 20 in other examples. The nut 36 can be formed of disposable materials, such as a disposable plastic. In this configuration, the nut 36 containing the first interference portion 25 is separate from the body 15 of the lens delivery device 10, although the nut 36 can be integrally formed with the body 15 in other examples.

FIGS. 2A-2C illustrate one example of the nut 36 that is configured to be positioned with the channel 20 of the body 15 of the lens delivery device. Referring more specifically to FIG. 2C, in this example, the first interference portion 25 is located within the nut 36. For example, the first interference portion 25 can be an o-ring located inside the nut 36 such that the first interference portion 25 can interact with the second interference portion 35 of the plunger 30 during axial movement of the plunger 30, as described in further detail below. In other examples, the first interference portion 25, such as an o-ring, can be located directly in the channel 20 of the body 15. The first interference portion 25, such as the o-ring shown in FIG. 2C, can be designed with various thicknesses or using materials of different flexibility to define the interaction with the second interference portion 35 based on the size of the lens 45 employed, as described in further detail below.

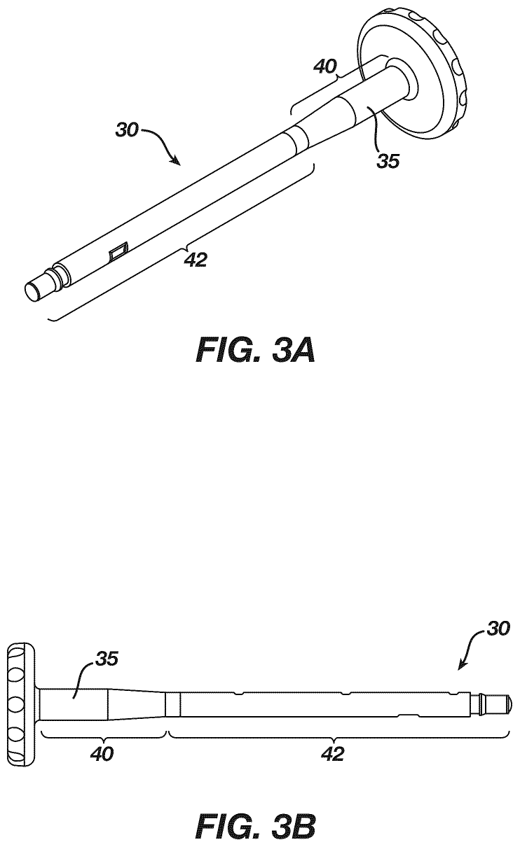

Referring now to FIGS. 1, 3A, and 3B, in this example the second interference portion 35 is located along a length 40 of the plunger 30. In this example, the second interference portion 35 is configured to interact with the first interaction portion 25, located either directly in channel 25 or in nut 36 (as shown in FIGS. 2A-2C, 4A-4C, and 6A-6C) to form the damping mechanism 12. The second interference portion 35 is located on the plunger 30 to interact with the first interference portion 25 based on a location of the lens 45 within the modular cartridge 50 during axial movement of the plunger 30. In some examples, the length 40 of the second interference portion 35 is approximately twice the optic diameter of the lens 45. In other examples, the length 40 of the second interference portion 35 is approximately 14 mm to 16 mm. In some examples, the second interference portion 35 is configured to interact with the first interference portion 25 until the lens 45 is ejected from the modular cartridge 50 as a result of the axial force applied to plunger 30. In other examples, the second interference portion 35 is configured to interact with the first interference portion 25 until the plunger 30 reaches an end-point of axial movement, along axis A (as shown in FIG. 1), through the channel 20 of the body 15. As shown in FIGS. 3A and 3B, for example, the second interference portion 35 is a portion of the plunger 30 having an increased diameter (along length 40) that causes the second interference portion 35 to interact with the first interference portion 25, such as the o-ring shown for example in FIG. 2C. The lower diameter along the length 42 of the plunger 30 is configured so that the plunger 30 does not interact with the first interference portion 25 along length 42 to allow for axial movement of the plunger 30 with less resistance.

As noted above, the dampening mechanism 12 can be used to provide the user with a consistent tactile feel as the user actuates (i.e. pushes) the plunger 30 to deliver the IOL from the modular cartridge 50 into the patient's eye. During the beginning of the delivery of the lens 45, the lens 45 is moved axially from the lens storage portion 52 toward the insertion tip 55 based on force applied from the pushrod tip 34. During the axial movement of the lens 45, the lens 45 is folded from a flat shape to a more cylindrical shape, as the diameter of the unfolded lens is much greater than that of the insertion tip 55. This folding action provides a resistance to the user operating the plunger 30. As the lens 45 progresses distally along the axis A into the modular cartridge 50 and closer to the insertion tip 55, the lens 45 begins to elongate along axis A, providing additional/consistent resistance to the user operation of the plunger 30. Once the largest portion of the folded and elongated lens 45 passes through the insertion tip 55, the resistance is suddenly lessened. The dampening mechanism 12 can be configured to approximately maintain the resistance on the plunger 30 after the resistance generated by the interaction between the lens 45 and the insertion tip 55 is dissipated by the ejection of the lens 45 therefrom. This dampening resistance provided by the damping mechanism 12 can be maintained for a finite time/distance after the resistance generated by the interaction between the lens 45 and the insertion tip 55 is dissipated, or alternatively, the damping resistance provided by the damping mechanism 12 can remain for the remaining stroke of the plunger 30 in the axial direction. Further, the dampening resistance provided by the damping mechanism 12 can be initiated at any point along the length of the plunger 30 or any position of the lens 45 within the modular cartridge 50.

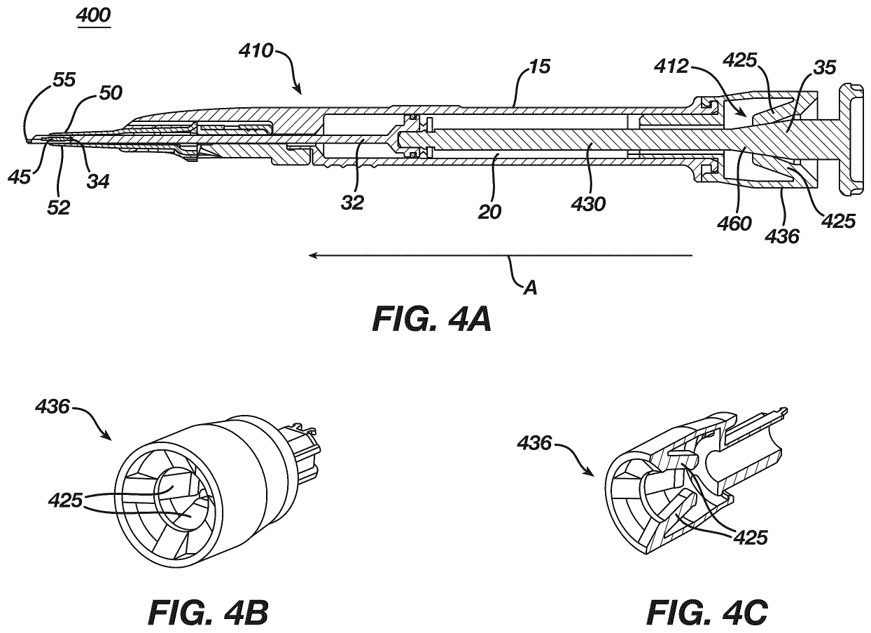

FIG. 4A illustrates another example of a system 400 for delivering the lens 45, such as an IOL, to a patient's eye including a lens delivery device 410 and the modular cartridge 50. The system 400 is the same in structure and operation as the system 100, except as described below. Like elements are illustrated and described using the same reference numerals. In this embodiment, the lens delivery device 410 includes a damping mechanism 412 that includes a first interference portion 425, which is located within a nut 436. The first interference portion 425 in this example, is provided by a set of flexible tabs extending from an inner surface the nut 436. In one example, the first interference portion 425 includes four flexible tabs extending from the inner surface of the nut 436, although other numbers of flexible tabs can be used. In this example, the first interference portion 425 interacts with the second interference portion 35 of the plunger 430 during axial movement of the plunger 430, as described above. In other examples, the first interference portion 425, such as the flexible tabs, can be located directly in the channel 20 of the body 15.

Referring more specifically to FIGS. 4B and 4C, in this example, the second interference portion 35 of the plunger 430 can cause the first interference portion 425 (flexible tabs) to bend to provide a damping force to resist the axial movement of the plunger 430 during operation based on friction between the first interference portion 425 (flexible tabs) and the second interference portion 35. The flexible tabs extend inward from the inner surface of the nut 436 and in a direction toward the insertion tip 55. The first interference portion 425, such as the flexible tabs shown in FIGS. 4B and 4C, can be designed with various thicknesses or using materials of different flexibility to define the interaction with the second interference portion 35 based on the size of the lens 45 employed, as described above.

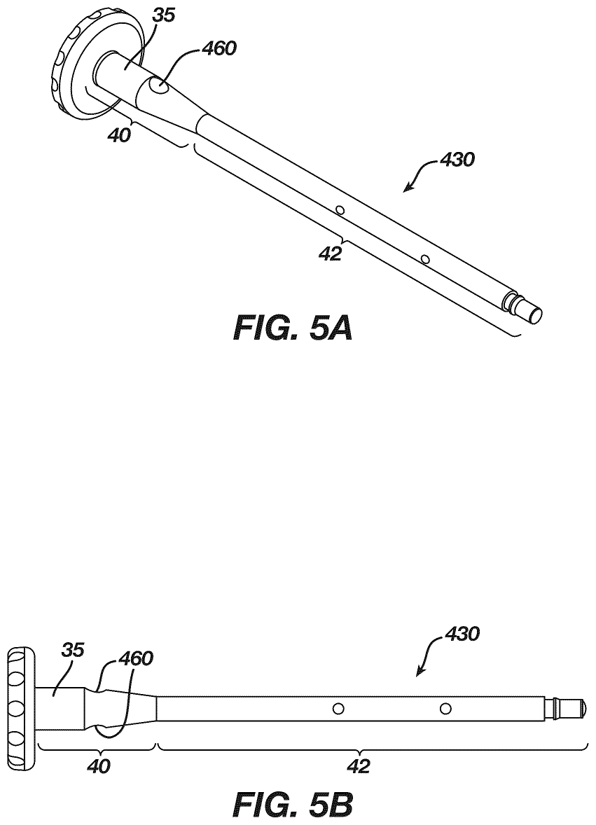

FIGS. 5A and 5B illustrate the plunger 430 illustrated in FIG. 4A, which is employed with the first interference portion 425 (flexible tabs) shown in FIGS. 4A-4C. In this example, the plunger 430 includes one or more recesses 460 that are positioned on the second interference portion 35. The recesses 460 are configured to “catch” at least a part of the first interference portion 425 (flexible tabs shown in FIGS. 4A-4C) at the end of the deployment, i.e., the full travel of plunger 430 to dispense the lens 45. The recesses 460 can be located at discrete locations on the second interference portion 35 to interact individually with the flexible tabs. Alternatively, the recesses 460 can be formed as a single recess that extends around the periphery of the second interference portion 35. The recesses 460 catch the first interference portion 425 (flexible tabs) to prevent retraction of the plunger 430 due to the force provided by the flexible tabs when the user-applied force is removed from the plunger 430.

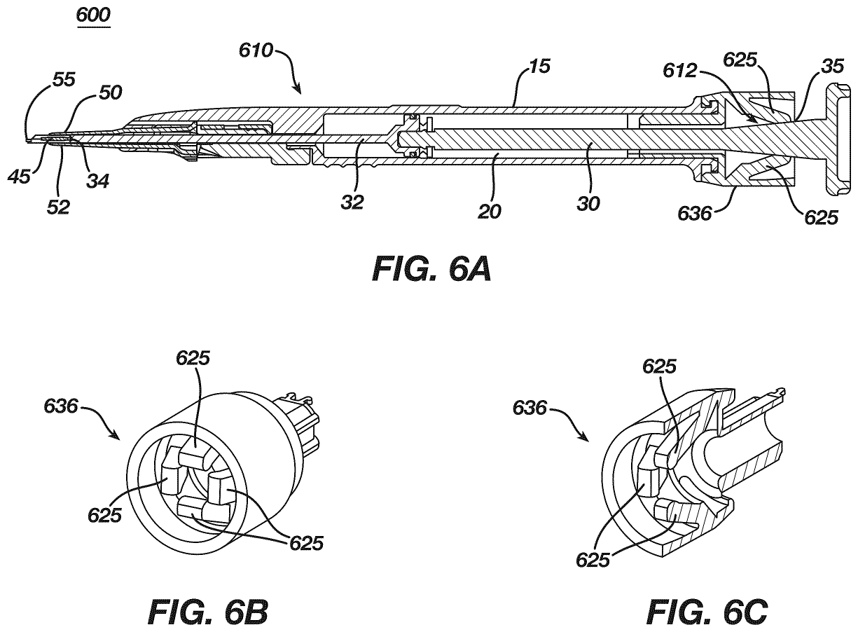

FIG. 6A illustrates another example of a system 600 for delivering the lens 45, such as an IOL, to a patient's eye including the lens delivery device 610 and the modular cartridge 50. The system 600 is the same in structure and operation as the system 100, except as described below. Like elements are illustrated and described using the same reference numerals. In this embodiment, the lens delivery device 610 includes a damping mechanism 612 that includes a first interference portion 625, which is located within a nut 636. In this embodiment, as shown in FIGS. 6A-6C, the first interference portion 625 is provided by a set of flexible tabs that extend from an inner surface the nut 636 and in a direction away from the insertion tip 55. In one example, the first interference portion 625 includes four flexible tabs extending from the inner surface of the nut 636, although other numbers of flexible tabs can be used. The flexible tabs operate in the same manner as described above, i.e., the flexible tabs interact with the second interference portion 35 of the plunger 30, such that the second interference portion 35 can cause the flexible tabs to bend to provide a damping force to resist the axial movement of the plunger 30 during operation based on friction between the first interference portion 625 (flexible tabs) and the second interference portion 35. The first interference portion 625, such as the flexible tabs shown in FIGS. 6B and 6C, can be designed with various thicknesses or using materials of different flexibility to define the interaction with the second interference portion 35 based on the size of the lens 45 employed, as described above. In this example, the flexible tabs are self-locking and the catch features, such as recesses 460 shown in FIGS. 5A and 5B, are not needed to prevent reverse movement of the plunger 30 after operation.

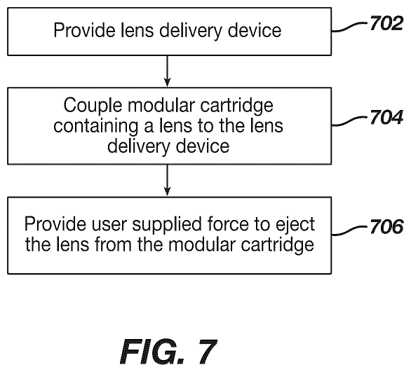

FIG. 7 provides a flowchart of an example method 700 for lens delivery.

In block 702, the method 700 can include providing a lens delivery device, such as lens delivery device 10. Although the exemplary method 700 is described with respect to lens delivery device 10, it is to be understood that the exemplary method 700 could be employed with other lens delivery devices, such as lens delivery devices 410 and 610, by way of example only. As discussed herein, the lens delivery device 10 can include a body 15 comprising a channel 20 therethrough. The lens delivery device 10 can further include a first interference portion 25 located within the channel 20. In some examples, the first interference portion 25 can be located in a separate nut 36 that is located in the channel 20, although in other examples, the first interference portion 25 can be located directly in the channel 20. The lens delivery device 10 can further include a plunger 30 configured to receive a user-supplied force to move the plunger 30 axially, for example along axis A as shown in FIG. 1, through the channel 20 of the body 15. The plunger 30 can include a second interference portion 35, located along a length 40 of the plunger 30 (as shown in FIGS. 3A and 3B, for example). The second interference portion 35 can be configured to interact with the first interference portion 25 during the axial movement of the plunger 30 to form a damping mechanism 12 to maintain a specified resistance to the user-supplied force, as described above.

In block 704, the method 700 can include coupling a modular cartridge 50 containing a lens 45, such as an intraocular lens, to the body 15. The axial movement of the plunger 30 is configured to drive the lens 45 through modular cartridge 50 into an insertion tip 55 to deliver the lens 45, such as an IOL, from the lens delivery device 10 to a patient's eye, as described above.

In block 706, the method 700 can include a user, such as a surgeon, providing a force to move the plunger 30 axially, for example along axis A shown in FIG. 1, through the channel 20 of the body 15 to eject the lens 45 from the modular cartridge 50 for insertion in the patient's eye. The user-supplied force on plunger 30 activates pushrod 32, which includes a pushrod tip 34 that moves the lens 45 from the lens storage portion 52 to the insertion tip 55. The constraining of the lens 45 in the insertion tip 55 causes the lens 45 to provide a force opposite to the user-supplied force on the plunger 30. Providing the user-supplied force on the plunger 30 causes the first interference portion 25 to interact with the second interference portion 35 prior to ejection of the lens 45 from the modular cartridge 50 to counteract the removal of the constraining force generated by the interaction of the lens 45 and the insertion tip 55 to provide for more controlled delivery of the lens 45, for example an IOL, to a user's eye. In some examples, the second interference portion 35 is configured to interact with the first interference portion 25 until the lens 45 is ejected from the modular cartridge 50 as a result of the axial force applied to plunger 30. In other examples, the second interference portion 35 is configured to interact with the first interference portion 25 until the plunger 30 reaches an end-point of axial movement, along axis A as shown in FIG. 1, through the channel 20 of the body 15.

The descriptions contained herein are examples of embodiments of the invention and are not intended in any way to limit the scope of the invention. As described herein, the invention contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

What is claimed is:

1. A lens delivery device comprising:
a body comprising a channel therethrough;
a first interference portion located within the channel; and
a plunger configured to receive a user-supplied force to move the plunger axially through the channel of the body, comprising:
a second interference portion, located along a length of the plunger, configured to interact with the first interaction portion to form a damping mechanism during the axial movement of the plunger to maintain a specified resistance to the user-supplied force.

2. The lens delivery device of claim 1, wherein the second interference portion is located on the plunger to interact with the first interference portion based on a location of the lens within the modular cartridge.

3. The lens delivery device of claim 2, wherein the second interference portion is configured to interact with the first interference portion until the lens is ejected from the modular cartridge.

4. The lens delivery device of claim 2, wherein the second interference portion is configured to interact with the first interference portion until the plunger reaches an end-point of axial movement through the channel of the body.

5. The lens delivery device of claim 1, wherein the first interference portion is an o-ring.

6. The lens delivery device of claim 1, wherein the first interference portion is a set of flexible tabs located within a circumference thereof of the body.

7. The lens delivery of device of claim 6, wherein the set of flexible tabs extend in a direction toward an insertion tip of the lens delivery device.

8. The lens delivery device of claim 7, wherein the plunger further comprises an engagement portion configured to interact with one or more of the set of flexible tabs to prevent retraction of the plunger when the user-supplied force is removed.

9. The lens delivery device of claim 6, wherein the set of flexible tabs extend in a direction away from an insertion tip of the lens delivery device.

10. The lens delivery device of claim 1, wherein the second interference portion is a portion of the plunger having an increased diameter that causes the second interference portion to interact with the first interference portion.

11. A system for delivering an intraocular lens (IOL) to a patient's eye, the system comprising:
a modular cartridge containing the IOL; and
a lens delivery device configured to receive the modular cartridge, the lens delivery device comprising:
a body comprising a channel therethrough;
a first interference portion located within the channel; and
a plunger configured to receive a user-supplied force to move the plunger axially through the channel of the body, comprising:
a second interference portion, located along a first length of the plunger, configured to interact with the first interference during the axial movement of the plunger to form a damping mechanism to maintain a specified resistance to the user-supplied force, wherein the axial movement of the plunger is configured to drive the IOL through the modular cartridge coupled to the body, delivering the lens from the lens delivery device.

12. The system of claim 11, wherein the dampening mechanism is configured to provide a force counter to the user-supplied force to slow the axial movement of the plunger based on the user-supplied force.

13. The system of claim 11, wherein the second interference portion is located on the plunger to interact with the first interference based on a location of the lens within the modular cartridge.

14. The system of claim 13, wherein the second interference portion is configured to interact with the first interference portion until the plunger reaches an end-point of axial movement through the channel of the body.

15. The system of claim 11, wherein the first interference portion is an o-ring.

16. The system of claim 11, wherein the first interference portion is a set of flexible tabs located within a circumference thereof.

17. The system of claim 16, wherein the set of flexible tabs extend in a direction toward an insertion tip of the lens delivery device.

18. The system of claim 17, wherein the plunger further comprises an engagement portion configured to interact with one or more of the set of flexible tabs to prevent retraction of the plunger when the user-supplied force is removed.

19. The system of claim 16, wherein the set of flexible tabs extend in a direction away from an insertion tip of the lens delivery device.

20. The system of claim 11, wherein the second interference portion is a portion of the plunger having an increased diameter that causes the second interference portion to interact with the first interference portion.
